(19) United States
(12) Patent Application Publication
(10) Pub. No.: US 2010/0217096 A1
(43) Pub. Date: Aug. 26, 2010

(54) A HEALTH MONITOR AND A METHOD FOR HEALTH MONITORING

(76) Inventor: Reuven Nanikashvili, Ashdod (IL)

Correspondence Address: SONNENSCHEIN NATH & ROSENTHAL LLP, P.O. BOX 061080, WACKER DRIVE STATION, WILLIS TOWER, CHICAGO, IL, 60606-1080, US

(21) Appl. No.: 11/625,739

(22) Filed: Jan. 22, 2007

Related U.S. Application Data
(60) Provisional application No. 60/761,239, filed on Jan. 23, 2006.

(51) Int. Cl. A61B 5/00

(52) U.S. Cl. 600/301

(57) ABSTRACT

A method for health monitoring and a personal health monitor that includes: a band that comprises at least one biometric sensor; and a data connector adapted to fasten a first end of the band to a second end of the band, and is also adapted to be connected to a reception device such as to provide data to the reception device; wherein the data connector is coupled to the at least one biometric sensor.

### RELATED APPLICATIONS

This patent application is a nonprovisional and claims the priority of U.S. provisional patent application No. 60/761,239, filed Jan. 23, 2006, entitled “Device and Method for Retrieving Medical Information”, and incorporated herein by reference.

### FIELD OF THE INVENTION

The present invention relates generally to a health monitor and to a method for health monitoring.

### BACKGROUND

Methods and systems for personal health and wellness monitoring are continuously developing in recent years. More specifically, these methods provide for continuous monitoring of individual's health condition.

Patients who need continuous monitoring of their health condition, may need to return frequently to their health care provider's in order to perform routine tests, mainly aimed at measuring biometric parameters which may include body temperature, heart rate, blood pressure, ECG, glucose level and body fat analysis. The need for the patient to frequently return to the health care provider is inconvenient, time consuming and expensive.

Arrangement for home care by professional individuals such as nurses or medical doctors who visit patients in order to continuously monitor their health can also be expensive and inconvenient. Addressing this issue by placing a home-based equipment at the patient's site is often bulky and hard to operate, and as such also requires intervention of professional individuals.

Another area of concern is the need to routinely collect biometric parameters under patient's regular daily routine—either at rest, or during routine, or during intensive physical effort, typical to sport activity.

Yet another area of concern is the ability of the medical center to provide patients with important information on a timely manner. Such information may include scheduled reminders and notifications for drugs.

Yet another area of concern is the need for the medical center to provide for fast response in case of emergency. Such emergencies may include conditions of “no movement” of the monitored patient, response to falling of a patient, irregular changes in heartbeat rate, change in ECG and like.

The following U.S. patents and patent applications, which are incorporated herein by reference, provide a brief review of state of the art health monitoring systems and health monitoring methods: U.S. Pat. No. 7,018,339 of Birnbaum et al, titled “Method for processing heart rate information in a portable computer”; U.S. patent application 2004/0027244 of Menard, titled “Personal medical device communication system and method”; U.S. Pat. No. 5,390,238 of Kirk, et al., titled “Health support system”; U.S. Pat. No. 5,566,676 of Rosenfeldt et al., titled “Pressure data acquisition device for a patient monitoring system”; U.S. Pat. No. 5,772,586 of Heinonen et al., titled “Method for monitoring the health of a patient”; U.S. Pat. No. 5,840,020 of Heinonen et al., titled “monitoring method and a monitoring equipment”; U.S. Pat. No. 5,983,193 of Heinonen et al., titled “patient's nursing apparatus and nursing system”; US patent application 20060173712A1 of Dirk, titled “Portable medical information system”; US patent application 20050245793A1 of Hilton et al, titled “Personal wellness monitor system and process”; US patent application 70050203349A1 of Nanikashvili, titled “Personal health monitor and a method for health monitoring”; U.S. Pat. No. 6,366,871 of Geva titled “Personal ambulatory cellular health monitor for mobile patient”.

While various implementation of personal health monitoring solutions have been developed, there is a need for a design which encompasses the desired characteristic as presented in accordance with this technology.

### SUMMARY OF THE INVENTION

The invention provides a method for health monitoring, the method includes: (i) fastening a first end and a second end of a band wearable by a person using a data connector; (ii) gathering biometric data by at least one biometric sensor at least partially surrounded by the band; (iii) connecting the data connector to a reception device; and (iv) transmitting to the reception device data representative of the biometric data gathered by the at least one biometric sensor.

The invention provides a method for health monitoring, the method includes: (i) fastening a first end and a second end of a band wearable by a person using a connector; (ii) gathering biometric data by at least one biometric sensor at least partially surrounded by the band; and (iii) transmitting to a reception device data representative of the biometric data gathered by the at least one biometric sensor. The transmitting can include wireless transmitting the biometric data, transmitting the biometric data via a data connector, and the like.

The invention provides a personal health monitor that includes: (i) a band that includes at least one biometric sensor; and (ii) a data connector adapted to fasten a first end of the band to a second end of the band, and is also adapted to be connected to a reception device such as to provide data to the reception device; wherein the data connector is connected to the at least one biometric sensor.

The invention provides a personal health monitor that includes: (i) a band that includes at least one biometric sensor; and (ii) a connector adapted to fasten a first end of the band to a second end of the band; and (iii) a communication circuit adapted to transmit biometric data. The communication circuit can be at least partially surrounded by the band, connected to the band and the like. The communication circuit can wirelessly transmit biometric data, utilize a data connector to transmit biometric data via a wired connection and the like.

### BRIEF DESCRIPTION OF THE DRAWINGS

Further features and advantages of the invention will be apparent from the description below. The invention is herein described, by way of example only, with reference to the accompanying drawings, wherein:

FIG. 1 is a schematic functional block diagram of a health monitoring system, according to an embodiment of the invention;

FIGS. 2A-2C are simplified illustrations of a person that wears a personal health monitor, according to various embodiments of the invention;

FIGS. 3A and 3B are simplified illustrations of a medical bracelet, according to an embodiment of the invention;

FIG. 4 is a schematic block diagram of a personal health monitor, according to an embodiment of the invention;

FIGS. 5a-5e illustrates various screens displayed to a user according to an embodiment of the invention; and

FIG. 6 is a flow chart of a method for health monitoring, according to an embodiment of the invention.

### DETAILED DESCRIPTION

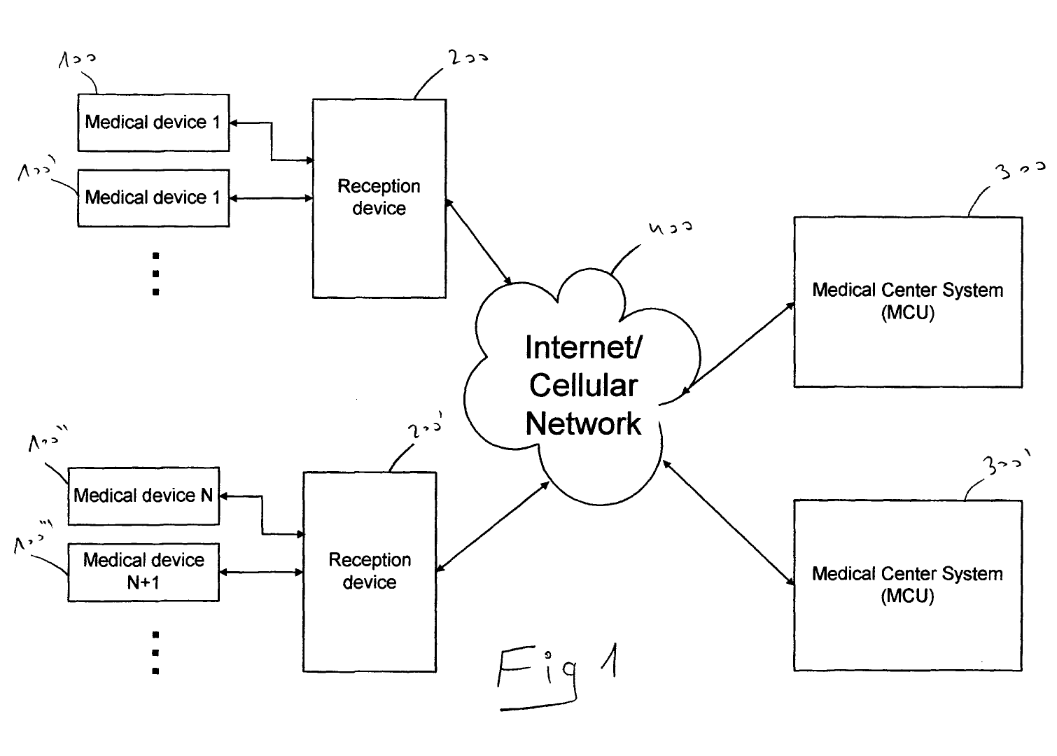

Reference is now made to FIG. 1, which shows a schematic functional block diagram of a system for personal health monitoring constructed and operative in accordance with an embodiment of the present invention.

Bracelet 100, which is attached to a monitored person, is configured to perform biometric parameters measurement on that person, to process the biometric data and transmit them via link 120 to a reception device 200. Link 120 may be a wireless connection such as WiFi, Bluetooth, cellular, or a wired connection such as a direct USB connection.

Link 120 may also be used to transmit data from reception device 200 to bracelet 100. Such data may be, but not limited to, setup parameters of bracelet 100. Reception device 200 may be a laptop computer, a desktop computer, a Personal Digital Assistant (PDA), a pocket PC, a cellular phone and like.

Reception device may further connect to medical center System 300, conveniently, via Internet connection or cellular network 400. Medical center System 300 is conveniently, constructed of a computer system with a Database and dedicated computer software. Medical Center System is also conveniently located in as medical clinic such as a hospital.

It is noted that more than one bracelet can be connected to a single reception device, and more than one reception device can be connected to a single medical center. With reference to FIG. 1, by way of example, both bracelets 100 and 100′ are connected to reception device 200, while bracelets 100″ and 100′″ are connected to reception device 200′.

It is also noted that a single reception device may be connected to more than one medical center. With reference to FIG. 1, by way of example, both reception devices 200 and 200′ can connect to either medical center system 300 or medical center system 300′.

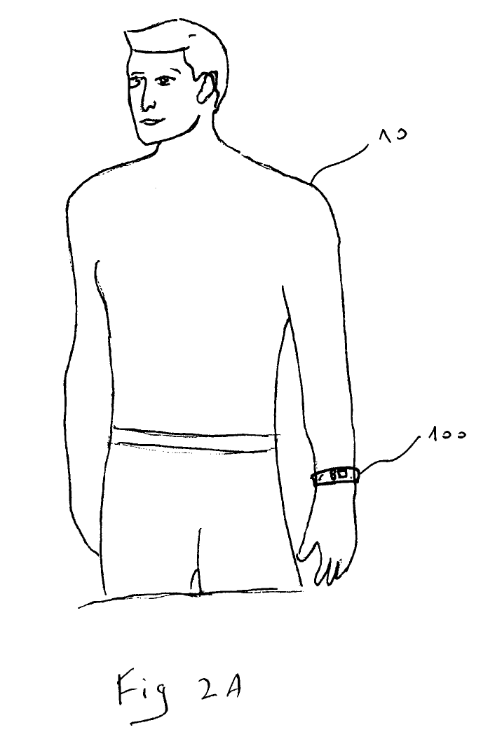

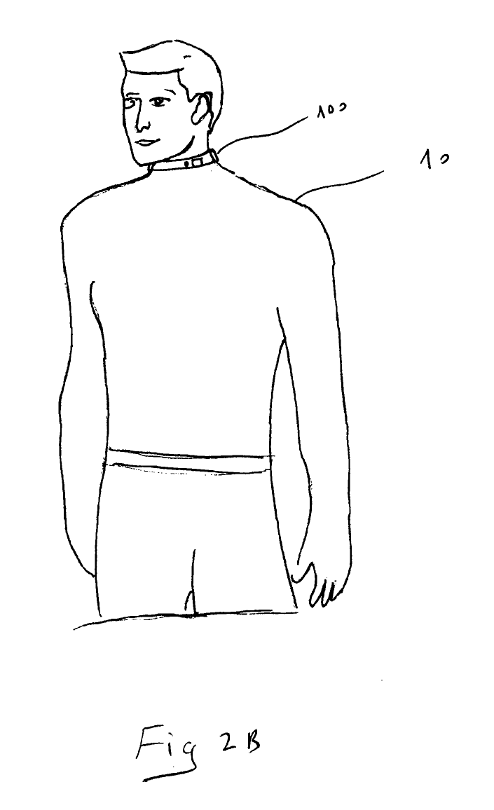

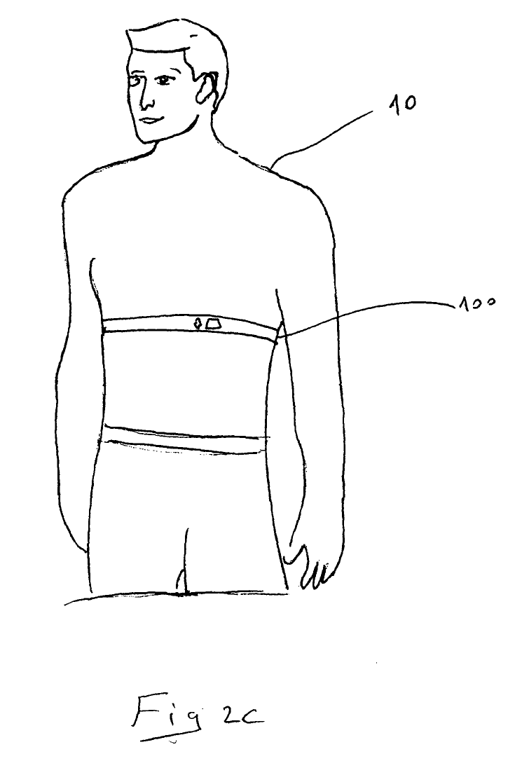

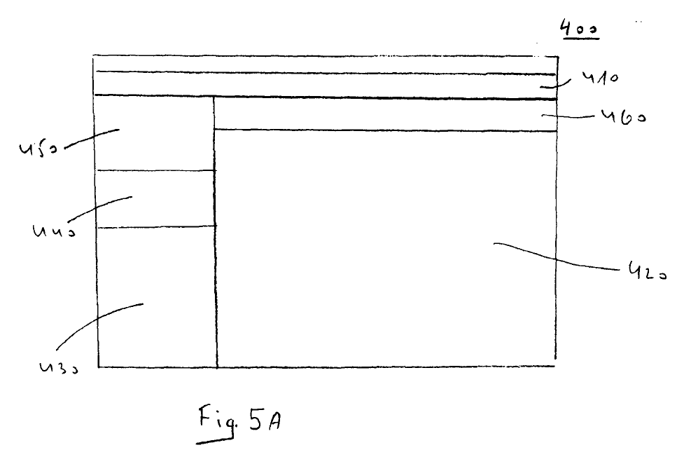

Reference is made now to FIG. 2A, which shows a simplified illustration of a bracelet in accordance with one embodiment of the present invention. Monitored person 10 carries a bracelet 100 which has the form of a bracelet. The monitored person can also use a display and an operating buttons (both illustrated in FIG. 4) to set up the bracelet, and to retrieve and send information.

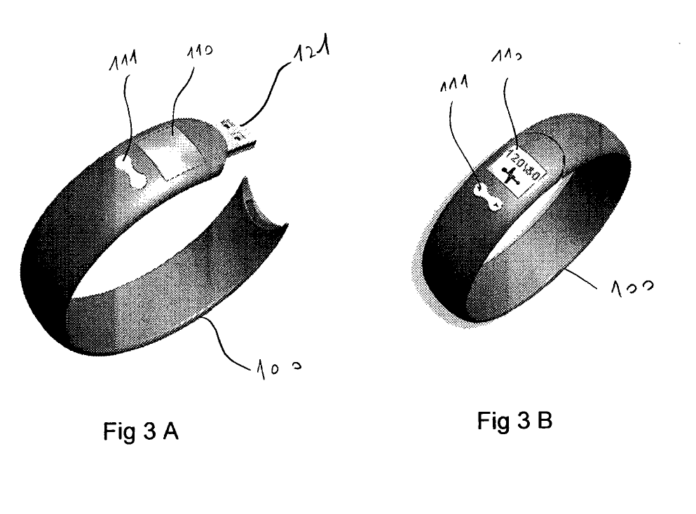

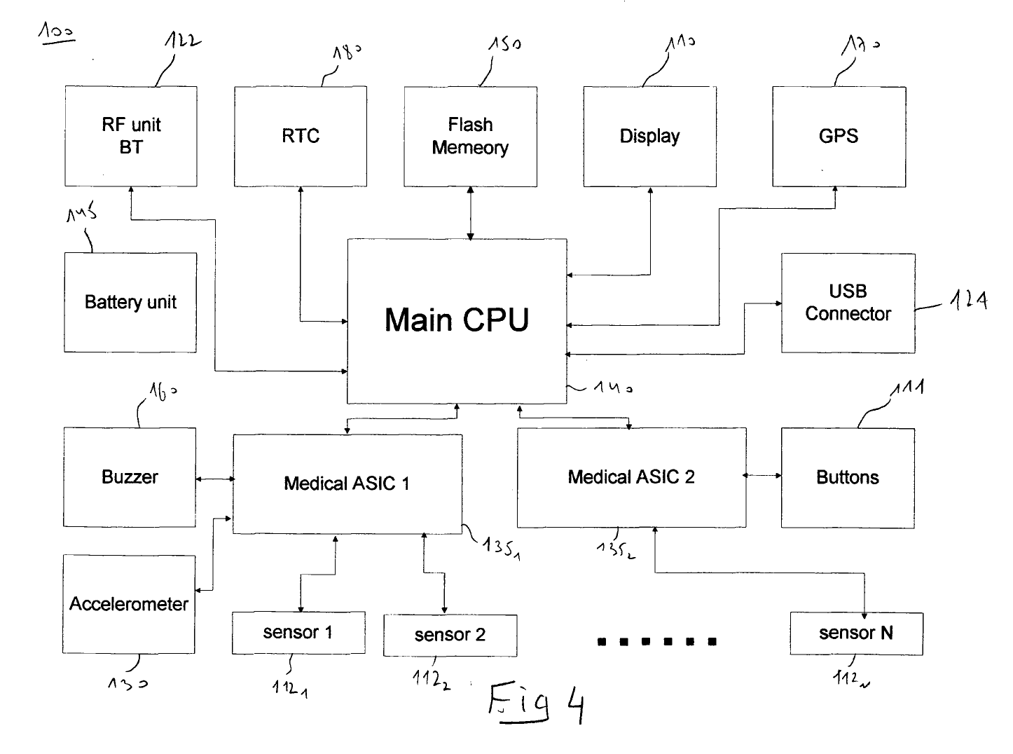

Reference is now made to FIGS. 3A and 3B which show a simplified illustration of a personal health monitor such as bracelet 100.

A data connector such as USB connector 121 is adapted to enable direct connection to reception device 200, as a link 120 shown in FIG. 1, and may be also used in as a physical connection which buckles the bracelet to the arm of the monitored person 10.

It is noted that although FIG. 2A illustrates a bracelet that is worn on the hand of a monitored person that the bracelet can also be worn on the leg of a monitored person (for example, near his ankle).

With reference to FIGS. 2B and 2C forms other than a bracelet may be used. Such forms include a necklace, as shown in FIG. 2B, and a chest belt, as shown in FIG. 2C. It is appreciated that other form factors may be used, in addition to those illustrated in FIG. 2A-2C.

It is appreciated, therefore, that whenever the term “bracelet” (or “health bracelet”) is used, it may also include other forms of the personal health monitor that include a band and a buckle, such as, but not limited to, a necklace, a chest belt and like.

Reference is now made to FIG. 4, which illustrates a simplified block diagram of bracelet 100, according to an embodiment of the present invention. The bracelet 100 includes sensors, 112₁ to 112_{N}, adapted to measure biometric parameters of the monitored person.

This set of sensors may include the following sensors: Heartbeat rate meter, One lead EGO, Glucose level meter, Body temperature meter, Ambient temperature meter.

Bracelet 100 may also include an accelerometer 130. The accelerometer readings and optionally some of the sensed biometric information) can be used to: (i) detect when the person fall (rapid acceleration of bracelet, while the bracelet is still worn by the person and still senses biometric signals), (ii) determine when the person does not move, (iii) pedometer application, and the like.

It is appreciated that the embodiment is not limited to any set of specific sensors or number of parameters and other or additional sensors may be added accordingly.

The control, processing and storage of measured data are processed by Medical ASICs 135₁ and 135₂, and the Central Processing Unit (CPU) 140, which also controls the whole operation of the bracelet 100. The processed measured data is stored within Flash memory 150, which can be further transmitted to reception device 200, either via direct connection, such as USB connection 121, or via wireless connection 122. Wireless connection may be either of the following: WiFi transmission, cellular communication, short range Bluetooth and like.

Bracelet 100 also include display 110 and buttons 111, which are used by the user for performing certain tasks, such as setting up the bracelet, retrieving information sent to the user from the reception device 200, and sending data to reception device 200.

Buzzer 160 is used to provide the user with specific indications, such as a reminder and notification for drugs.

Bracelet 100 can also control room air temperature, using RF link 112 to an air conditioning system (not shown). The control can be set manually by the user or, alternatively, it can be responsive to the status of the monitored person (heart beat, perspiration, body temperature), but this is not necessarily

Bracelet 100 may be also configured to play MP3 music files stored in Flash Memory 150, conveniently, using a Bluetooth headset connected to bracelet 100 via Bluetooth link 122.

Bracelet 100 also includes a GPS unit 170, which is used to measure and store accurate positions of the monitored person. The position information may be used to locate the monitored person in case of emergency.

Also included in Bracelet 100 a Real Time Clock (RTC) which is used to perform time dependent tasks, such as drug reminder, monitoring patient's location vs. time and like.

Also included in the Bracelet 100 is a battery 145. This battery may be either a rechargeable type or a replaceable type.

The reception device 200 is now described. The reception device 200 can receive medical signals from multiple Bracelets; 100, 100′, etc. as shown in FIG. 1. The reception device may be realized on a laptop computer, Personal Digital Assistant (PDA), pocket PC, desktop computer, smart cellular phone and like. By way of example, the following description refers to realization of the reception device on a laptop or a desktop computer, but it is appreciated that equivalent realizations may be on other computing platforms.

The reception device 200 executes a software for retrieving the medical information, processing the information as well as additional tasks. This software launches automatically when the bracelet is connected to the reception device 200 via the USB connector, or executed manually when other connection methods are applied.

The software can add, edit and view demographic information, medical information, etc. The desktop application optionally has connectivity to local database (conveniently, an XML based DB).

The software user interface is a Graphical User Interface (GUI) that can be used by a doctor or clinician in MCS 300 or 300′, or by a user of reception device 200 or 200′.

The software is available in two installation configuration (i) a stand alone configuration for reception device 200 or 200′, and (ii) a Web center configuration which is most suitable for Medical Center System (MCS) 300 or 300′. It is noted that all software features detailed below are available for both configurations.

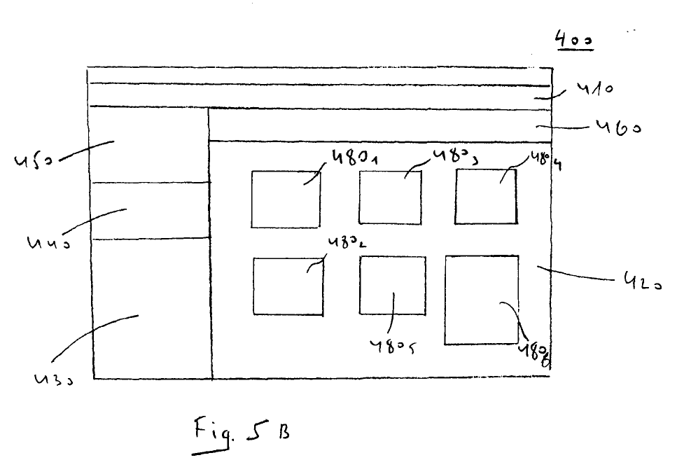

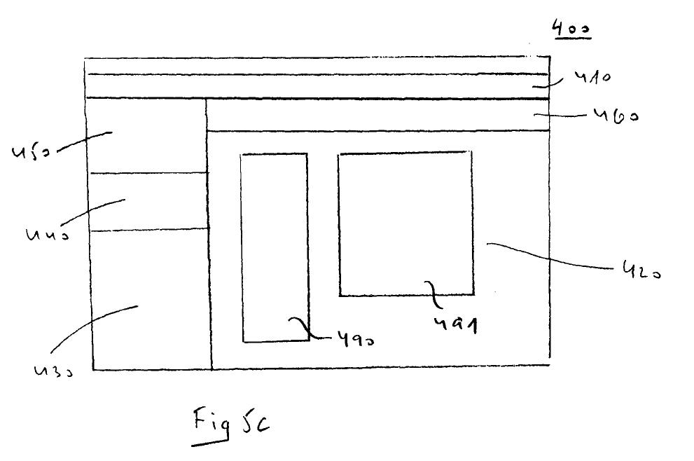

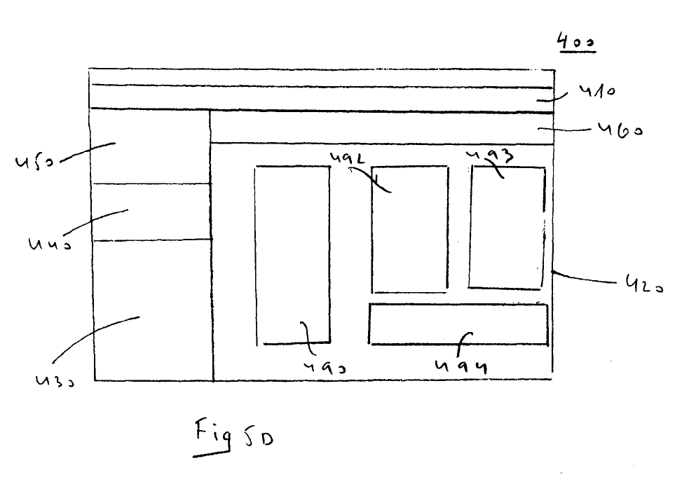

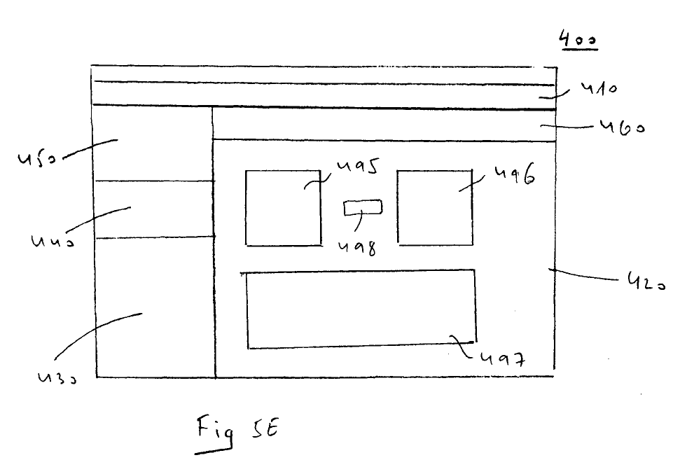

The software features and operation will now be explained, with reference to FIGS. 5A-5E.

Reference is now made to FIG. 5a, which illustrates the general Graphical User Interface (GUI) used to operate the software. The design of the desktop screen 400 may resemble Outlook 2005 type of interface.

The top side 410 of screen 400 contains a dynamic task bar, which varies dynamically according to the specific screen selection. The left hand side of the screen contains various buttons and lists or search fields. Field 450 is a fixed display of a list of patients, field 440 contains a fixed patient search control which allows user to select specific patient according to specific filled search fields. This provides the starting point of the application. 430 is a Tab Selected Functionality field, which may be used to navigate between the various screens, detailed below.

The right hand side of the screen 420 is a data working area which contains the detailed information or other entities which are specific for each page. Also shown is a User Warning Area 460 which is used to display warnings to user such as lack of communication channel, warning of attempt to send an empty file, etc.

Reference is now made to FIG. 5b, which illustrates the patient page.

The page allows the user to add or view details of a new patient, or an existing patient after completing the searching operation (first in a local database and then in a central database). Patients field may include patient name details (480₁), Address field (480₂), Birth data field (480₃), Gender data field (480₄), Contact data field (480₅) which may include data such as phone numbers and email. Medical data field 480₆ may contain data such as smoking record, height and weight, and like. It is noted that these fields are shown only by way of example, and different fields may be used.

Working area 420 of the receive page (not shown) contains a list of all sets of available exams performed on a specific patients. The user can scroll through all historical data by pressing either the “previous” or “next” buttons located at the Tab Selected Functionality field 430.

Reference is now made to FIG. 5C which shows an example of a Test page, which allows the user to perform live tests on a patient.

The working area 420 of the test page contains a test selection field 490, which is used to select the specific test to be performed (Blood pressure, ECG, etc.) and a specific test field 491 which is used to display the test results. The test is initiated and concluded using “start” and “stop” buttons located at Tab Selected Functionality field 430.

Reference is now made to FIG. 51y which shows the medical history page. This page allows the user to view all tests performed on a patient. Field 490 allows the user to select a specific test, field 492 displays test results, field 493 displays additional information of a specific test, such as experts' comments and advices. Field 494 contains details on medical tests transmission.

Tab Selected Functionality field 430 allows the user to perform additional tasks such as displaying graphs of medical tests, data printing and deleting, etc.

Send page (not shown) allows the user to send a whole set of exams performed on one patient to Medical Center System 300. Tests records are displayed on working area 420, and “send” or “resend” operation are performed using corresponding buttons located at Tab Selected Functionality field 430.

Setup page (not shown) allows the user to setup the system. Two types of setups are available: (i) General setup which allows user to configure general system parameters, and (ii) Medical setup which allows user to setup bracelets parameters. Setup data is further seat to a specific user bracelet 100 via link 120 in FIG. 1.

Reference is now made to FIG. 5E which illustrates the Drug reminder page.

The page allows the user to configure the settings for the drug reminder, which configures the medication name, dosage, quantity and time in which the patient should take the medication. Field 495 contains a list of available medications, while field 496 contains a list of selected medications for the current patient.

Medications can be added or removed by using the “add/remove” button 498. Field 497 contains the reminder data, such as the scheduled hours for each medication, dosage/quantity, etc. Drug reminder data is further sent to a specific user's bracelet 100 via link 120. Reminder function notifies the user by buzzer 160 patient's need for medications. Details of drug (i.e. name and dosage) are displayed on screen 110.

The health monitor may execute various types of software as well as use various interfaces.

The software application can use XML based database to save the new or edited data. This applies to the data base of reception device 200, as well as to the database of Medical Center System 300. The database contains information regarding: Subscribers (patients) demographics, Devices, Events (ordered according to Lot's—batches of various medical tests, performed at a certain date), Other settings (user name, password, etc.), clinics demographics, and software for use at the reception device 200. This software is suitable for use at a software professional site, such as a stand alone clinic. This software also includes connectivity to Medical Center System 300.

The software can include software for use at the Medical Center System 300, which is typically located at a Web center client, such as a hospital.

The software supports abilities to connect/synchronize with other medical devices

Reading medical data from the USB based bracelet, viewing it on PC and sending data to medical center system 300. It also enables retrieving of medical personal file of the patient, and synchronizing data with the medical center system 300. It also enables the configuration of the drug reminder on the bracelet 100.

The software is automatically launched whenever the bracelet is connected to the USB port.

The software medical system requires high security levels of consideration.

The internet messages uses secured HTTP (https://) with proper certificate authorization (CA) from a known CA authority.

Software shall have a secured login process that will prevent un-authorized people to view private medical data.

It also allows access to database sources only through the applications and prevents un-authorized access to database through the database viewing and editing tools.

The software application can use internet XML http requests to store and fetch data in the software web center database.

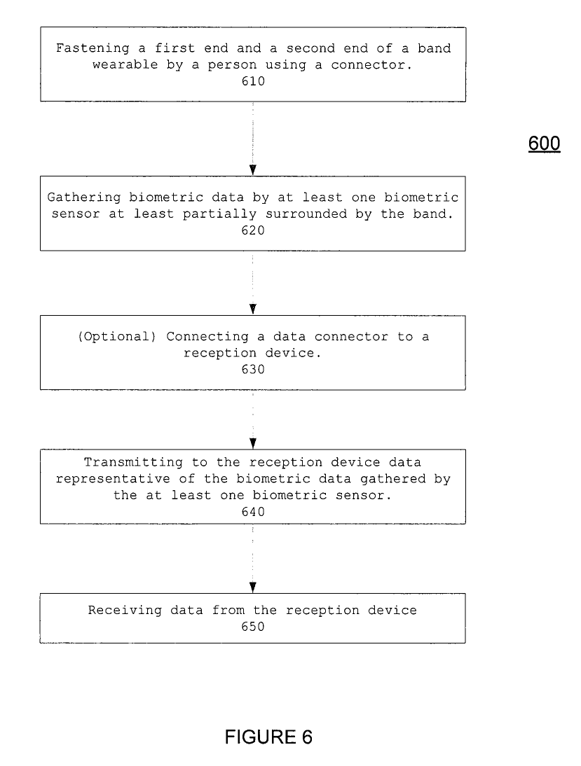

FIG. 6 is a flow chart of method 600 according to an embodiment of the invention.

Method 600 starts by stage 610 of fastening a first end and a second end of a band wearable by a person using a connector such as a data connector.

Conveniently, stage 610 includes using a USB compliant data connector, fastening the first and second ends of the belt using a data connector, wherein the belt and the data connector form a bracelet, a necklace, a chest belt and the like.

Stage 610 is followed by stage 620 of gathering biometric data by at least one biometric sensor at least partially surrounded by the band.

Conveniently, stage 620 involves gathering of heartbeat data or using any of the previously mentioned sensors.

Stage 620 is followed by optional stage 630 of connecting a data connector to a reception device.

Optional stage 630 is followed by stage 640 of transmitting to the reception device data representative of the biometric data gathered by the at least one biometric sensor. The transmitting can utilize a data connector but can also involve wireless transmission of the biometric data using a wireless communication circuit of medical monitor. The medical monitor also includes the at least one biometric sensor.

Conveniently, stage 640 involves utilizing a wireless data link.

Conveniently, stage 640 is followed by stage 650 of receiving data from the reception device. The received data can include control and/or configuration information that can affect the operation of the monitor.

Those skilled in the art will readily appreciate that various modifications and changes may be applied to the preferred embodiments of the invention as hereinbefore exemplified without departing from its scope as defined in and by the appended claims.

What is claimed is:

1. A personal health monitor comprising:
a band that comprises at least one biometric sensor; and
a data connector adapted to fasten a first end of the band to a second end of the band, and is also adapted to be connected to a reception device such as to provide data to the reception device; wherein the data connector is coupled to the at least one biometric sensor.

2. The health monitor of claim 1 wherein the data connector is a USB compliant connector.

3. The personal health monitor of claim 1 wherein the band and the data connector have a form of a bracelet.

4. The personal health monitor of claim 1 wherein the band and the data connector have a form of a necklace.

5. The personal health monitor of claim 1 wherein the band and the data connector have a form of a chest belt.

6. The personal health monitor of claim 1 further comprising a display.

7. The personal health monitor of claim 1 further comprising at least one button.

8. The health monitor of claim 1 wherein at least one biometric sensor comprises a heartbeat meter.

9. A health monitor of claim 1 further comprising a receiver that is adapted to receive information over a wireless data link adapted from a reception device.

10. A method for health monitoring, comprising:
fastening a first end and a second end of a band wearable by a person using a data connector;
gathering biometric data by at least one biometric sensor at least partially surrounded by the band;
connecting the data connector to a reception device; and
transmitting to the reception device data representative of the biometric data gathered by the at least one biometric sensor.

11. The method of claim 10 wherein the stage of fastening comprises using a USB compliant data connector.

12. The method of claim 10 wherein the stage of fastening comprises fastening the first and second ends of the belt using a data connector that form of a bracelet.

13. The method of claim 10 wherein the stage of fastening comprises fastening the first and second ends of the belt using a data connector that form a necklace.

14. The method of claim 10 wherein the stage of fastening comprises fastening the first and second ends of the belt using a data connector that form a chest belt.

15. The method of claim 10 wherein the stage of gathering biometric data comprises the gathering of heartbeat data.

16. The method of claim 10 wherein the stage of transmitting to the reception device is performed over a wireless data link.

17. The method of claim 10 further comprising a stage of receiving data from the reception device.

18. A personal health monitor comprising:
a band that comprises at least one biometric sensor;
a connector adapted to fasten a first end of the band to a second end of the band; and
a communication circuit, coupled to the at least one biometric sensor, adapted to transmit biometric data.

19. The personal health monitor according to claim 18 wherein the connector is a data connector that is adapted to be connected to a reception device such as to provide data to the reception device;
wherein the data connector is coupled to the at least one biometric sensor.

20. A method for health monitoring, comprising:
fastening a first end and a second end of a band wearable by a person using a connector;
gathering, biometric data by at least one biometric sensor at least partially surrounded by the band;
transmitting to a reception device data representative of the biometric data gathered by the at least one biometric sensor.
